(19) United States
(12) Patent Application Publication
(10) Pub. No.: US 2026/0048184 A1
(43) Pub. Date: Feb. 19, 2026

(54) BREAST PUMP AND SET OF VACUUM CURVES

(71) Applicant: MEDELA HOLDING AG, Baar (CH)

(72) Inventors: Danielle Prime, Bateau Bay (AU); Leon Mitoulas, Stirling (AU); Brian H. Silver, Woodstock, IL (US)

(21) Appl. No.: 18/805,268

(22) Filed: Aug. 14, 2024

(51) Int. Cl. A61M 1/06

(52) U.S. Cl. CPC A61M 1/0693; A61M 2205/50

(57) ABSTRACT

The present invention relates to a breast pump including a breast shield adapted to at least in part receive a breast of a lactating user, an aggregate for generating a cyclic suction profile according to vacuum curves for expressing milk from the breast, a milk container providing a reservoir for the expressed milk, a controller for controlling the aggregate.

### FIELD OF THE INVENTION

The present invention relates generally to a breast pump and a set of vacuum curves.

For example, it can relate to a process for initiating breastmilk from a mother very shortly after giving birth, most particularly in the instance of a premature infant, using a motorized, such as electrically driven, breast pump.

### BACKGROUND

Breast pumps for use by nursing mothers are well known. They allow the nursing mothers to express the breastmilk as necessary or convenient, and further provide collection of the breastmilk for later use. For some mothers, breast pumps may be a necessity, such as when the child has suckling problems, or if the mother has problems with excessive or deficient milk production, or soreness, deformation, or injury of the mammilla. Of particular instance herein is the situation confronting a mother where the infant is premature, and therefore is most always separated from the mother.

Electrically-driven breast pumps are commonplace, typically including a vacuum pump which has an electric motor that plugs into standard house current, and/or operates with battery power. Advantages of this type of pump are convenience, ready controllability and regulation of the vacuum, and in many instances the ability to pump both breasts at once.

Electrically-driven, motorized breast pumps generally have a driving mechanism for generating the vacuum (negative pressure) to be applied at the breast geared to a particular sequence, or curve, of vacuum or negative pressure increase (i.e. increasing suction), and then release. The cycling vacuum reproduces baby behavior and has at least a stimulation pattern and an expression pattern; examples of such patterns are reproduced in WO 2021 160 875, WO 2010 096 547 and WO 03 082 378. Each pattern is made of vacuum curves that are cyclically reproduced by the vacuum pump.

### Underlying Problem

Different mothers have different sensitivities and can endure different vacuum strengths for different application times; for this reason, usually vacuum pumps can reproduce not one single vacuum curve, but a set of vacuum curves comprising of many vacuum curves that differ in terms of vacuum strength and cycle length, i.e. duration of each cycle.

In light of this, prior art pumps allow the user to alter the vacuum strength by introducing a respective command via the user interface. Typically, a stronger vacuum strength (corresponding to a lower pressure) corresponds to a longer cycle length.

However, mothers who are sensitive to the strength of vacuum, maintain a lesser vacuum so reducing breast exposure to vacuum and thus milk expression.

The present invention aims to provide a breast pump and a method for operating the same which maximizes the vacuum exposure on the nipple and fulfills the user's need for a comfortable yet effective breast pumping.

### SUMMARY OF THE INVENTION

To cope with this problem, the present invention provides a breast pump including a breast shield adapted to at least in part receive a breast of a lactating user. The breast pump furthermore has an aggregate for generating a cyclic suction profile for expressing milk from the breast. The breast pump also has a milk container providing a reservoir for the expressed milk. Further, a controller is provided for controlling the aggregate. In data communication with said controller, there is provided an interface adapted to change an operational parameter of the aggregate.

The interface may be provided on a housing surrounding the controller and/or the aggregate. The interface may likewise be a wirelessly connected interface which is provided by a handheld such as a smartphone or tablet or the like.

The present invention allows an increase of milk extraction from the breast, because it was surprisingly found that application of the tolerable maximum vacuum strength may be limiting, for example, mothers who are vacuum sensitive and pumping with a comfortable vacuum that is less efficient compared to a stronger vacuum, if she was, indeed, tolerant of a stronger vacuum or for mothers who are tolerant of the strongest vacuum offered but could tolerate increase vacuum exposure. For example, in addition to milk flow, reduced production could be due to the reduced time moms inadvertently use breast pumps, and other factors, that as a whole make moms reduce time of using the breast pumps, etc.

For a more detailed analysis, vacuum curves have a cycle phase and a relaxation phase.

In the cycle phase, the first phase of a vacuum curve will be identified as a “vacuum rise phase”. This vacuum rise phase starts with relaxation pressure and reduces the pressure up to the maximum vacuum and so minimum pressure. The vacuum rise phase has a negative pressure gradient. After this, the minimum pressure may be maintained for a certain period of time, in a “vacuum peak phase”. This “vacuum peak phase” is followed by a “vacuum release 1 phase” or first vacuum release phase in which the vacuum strength is initially decreased, which vacuum release phase is followed by a vacuum hold phase in which the vacuum strength remains essentially constant. After this vacuum hold phase, vacuum strength is reduced to the relaxation pressure in a further vacuum release 2 phase.

After the cycle phase, the relaxation pressure is maintained during the high pressure vacuum relaxation phase; after the high pressure vacuum relaxation phase a new cycle phase starts.

According to a preferred embodiment, at least 70% of any variation of the cycle length is provided by the variation of the vacuum hold phase and/or the vacuum peak phase and/or the first vacuum release phase. Specifically, the vacuum hold phase and/or the vacuum peak phase and/or the first vacuum release phase will be used to either prolong or reduce the cycle length in response to a command entered by the user to alter the vacuum strength.

In a preferred embodiment of the present invention, the controller has a memory having stored therein a set of at least two, preferably at least three, most preferably at least four vacuum curves, which can be selected by the user to be applied. For example, a set of 16, 18, 20 or even more vacuum curves can be stored in the controller. In the set of vacuum curves, vacuum curves with a stronger or deeper maximum vacuum also have shorter cycle phase and vice versa, i.e. vacuum curves with weaker maximum vacuum have longer cycle phase. Preferably, vacuum curves with a stronger or deeper maximum vacuum also have shorter vacuum curve length and vice versa, i.e. vacuum curves with weaker maximum vacuum have longer vacuum curve length.

Other features of the invention will become apparent to those skilled in the art upon consideration of the following detailed description of preferred embodiments, exemplifying the best mode of carrying out the invention as presently received.

### DESCRIPTION OF THE DRAWINGS

These and other advantages of the invention will be further understood upon consideration of the following detailed description of certain embodiments, taken in conjunction with the drawings, in which:

FIG. 1 is an illustration of a breast pump assembly for use in accordance with one embodiment of the present invention;

FIG. 2 is a graphical representation of four measured suction profiles and

FIG. 3 is a schematic graph of different phases of the actual suction profile as e.g. shown in FIG. 2.

### DETAILED DESCRIPTION OF THE DRAWINGS

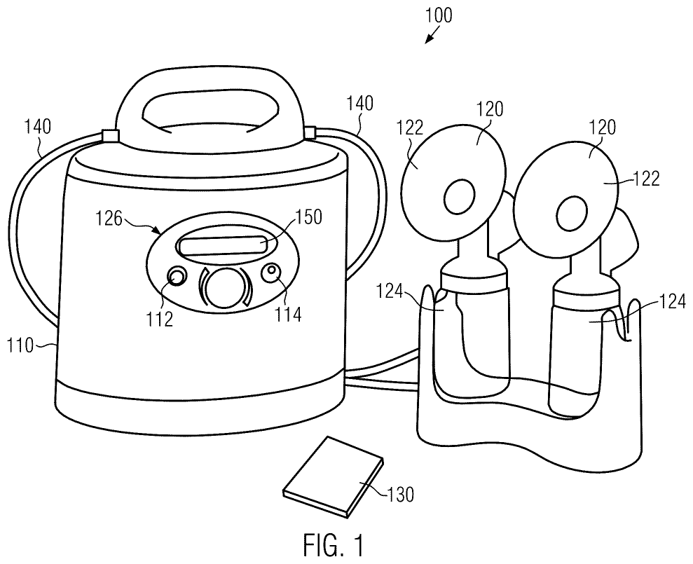

FIG. 1 is an illustration of a breast pump assembly in accordance with one embodiment of the present invention. That breast pump is generally described in U.S. Pat. No. 6,547,756 or EP 2 878 317 B1, reference thereto can be made for salient details of this breast pump. While the invention has found particular application for use with this kind of programmable breast pump and with respect to premature newborn, it can be used or adapted for use with other motorized pumps capable of being operated with the varying sequences (hereinafter described), and aspects are considered adaptable to full-term newborn including breast pumps wearable inside a bra, as disclosed in WO 2022/268998.

As shown in FIG. 1, the breast pump assembly 100 includes a breast pump 110, one or a plurality of the breast shield and container assemblies 120, which may provide a program card 130. Power may be provided to the breast pump apparatus 110 either through standard current via a power cord, a battery, or some other appropriate power supply.

The breast pump 110 may be either a double or a single pump. The single pump extracts milk from one breast at a time, and the double pump can be used to extract milk from both breasts at the same time. The breast pump 110 is attached to each of the plurality of the breast shield and container assemblies 120 with a tube 140. Each of the plurality of the breast shield and container assemblies 120 comprises a breast shield 122 and a container 124. The container 124 is used as a reservoir to store the pumped milk.

One significant aspect of the present invention is the ability to operate, as by program, the breast pump 110 with different types of cyclic suction profiles.

The breast pump 110 utilizes a controller 126 for controlling an aggregate, so that the aggregate generates the required cyclic suction profile. The controller has access (e.g. via a memory or because they are stored on the control itself) to vacuum curves that are indicative of vacuum over time to be generated by the aggregate and these vacuum curves are used by the controller to drive the aggregate.

The controller 126 or microprocessor-based system is provided with user input, for example through the program card 130.

To extract breastmilk from a mother, the breast shields 122 are placed and centered over a mother's nipples. The breast pump apparatus 110 may be turned on by a user pressing a first button 112, and in this embodiment, the program card 130 is used with the apparatus. The apparatus reads the program contained on the program card 130. The breast pump 110 may display instructions to the user via interface 150. The instructions may ask the user to start the program. If the mother wants to start the program, the mother may press a second button 114. The Interface 150 may then show instructions and/or graphics that let the mother know that the program is starting.

The mother can select via the interface e.g. the maximum vacuum strength she wants to use; each maximum vacuum strength is part of a vacuum curve that is accordingly selected. The controller thus drives the aggregate according to the selected vacuum curve.

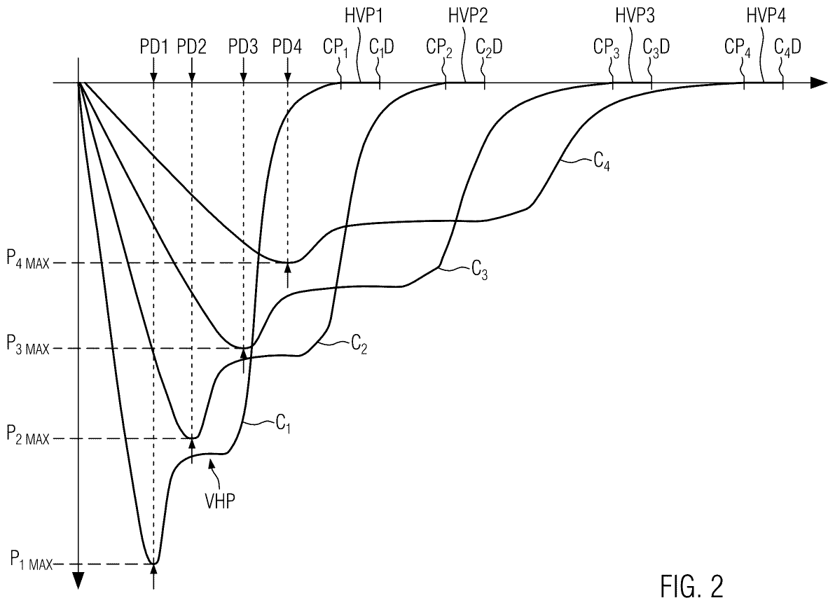

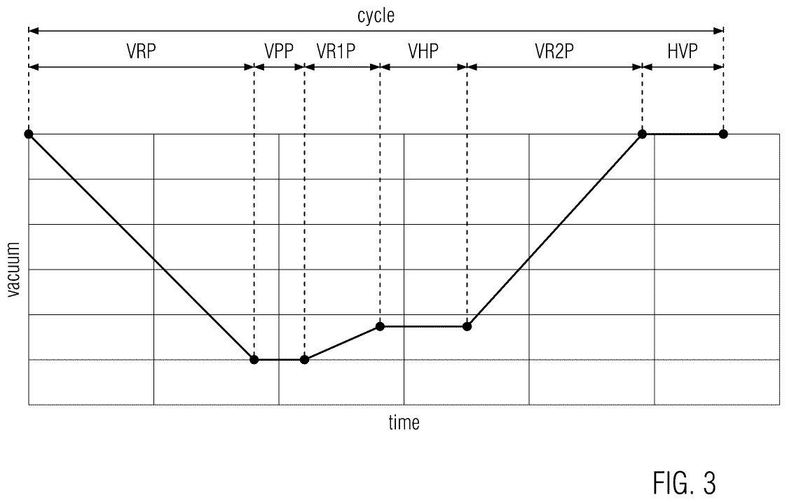

FIG. 2 shows different vacuum curves of a set of curves available to be chosen in a breast pump. Each vacuum curve of the set of curves has cycle phase C₁, C₂, C₃, C₄ and a high pressure vacuum relaxation phase HVP₁, HVP₂, HVP₃, HVP₄ (HVP in FIG. 3) at constant vacuum (or pressure); the relaxation phase is for example at atmospheric pressure, but it may also be at negative pressure. At the end of each vacuum curve and in particular relaxation phase thereof, the next vacuum curve starts.

This start begins at the horizontal timeline at zero, orthogonal to set abscissa, and starting at zero is an ordinate for the actual (negative) pressure. Each vacuum curve, and in particular the cycle phase thereof C₁, C₂, C₃, C₄, is characterized by a certain duration, i.e. cycle time CT, and a maximum vacuum strength PD. C₁ has the largest or strongest maximum vacuum strength PD₁ and provides the lowest minimum pressure. C₁ also has the shortest cycle phase duration, CT₁. C₄ has the longest cycle phase duration CT₄ and the weakest maximum vacuum strength (thus highest minimum pressure) PD₄.

Each cycle phase can be fractioned into different phases, which are set out in FIG. 3. The first phase is the vacuum rise phase VRP in which the vacuum increases and the pressure drops up to the strongest vacuum strength, i.e. maximum suction and thus minimal pressure. The strongest vacuum strength may be held for a certain, usually very short period of time. This phase is identified as the vacuum peak phase VPP. From maximum vacuum strength, the vacuum drops (and pressure increases) during the first vacuum release phase VR₁P to reach a vacuum hold phase VHP, in which the vacuum (negative pressure) is essentially constant for a longer period of time. From the vacuum hold phase VHP the vacuum drops (i.e. pressure rises) in the second vacuum release phase VR₂P until the relaxation pressure is again reached and held constant during the high pressure vacuum relaxation phase HVP.

FIG. 2 shows that, in the set of vacuum curves, the stronger the maximum vacuum strength, the shorter the cycle phase duration, e.g. the vacuum hold phase VHP and/or the vacuum peak phase VPP and/or the first vacuum release phase VR1P; likewise, the weaker the maximum vacuum strength P_{i max}, the longer the cycle phase duration, e.g. the duration of the vacuum hold phase VHP and/or vacuum peak phase VPP and/or first vacuum release phase VR1P. Preferably, in the set of vacuum curves, the stronger the maximum vacuum strength, the shorter the vacuum curve duration, e.g. the vacuum hold phase VHP and/or the vacuum peak phase VPP and/or the first vacuum release phase VR1P and/or the high pressure vacuum relaxation phase HVP; likewise, the weaker the maximum vacuum strength, the longer the vacuum curve duration, e.g. the duration of the vacuum hold phase VHP and/or the vacuum peak phase VPP and/or the first vacuum release phase VR1P and/or the high pressure vacuum relaxation phase HVP.

In addition, since preferably at least 70% of any variation of the cycle phase duration or vacuum curve duration is provided by the variation of the vacuum hold phase VHP and/or the vacuum peak phase VPP and/or the first vacuum release phase VR1P and/or the high pressure vacuum relaxation phase HVP, such a variation can be achieved without affecting or with limited affecting of the gradient of the vacuum rise phase VRP and/or vacuum release phases VR2P, which can thus be set according to the needs, e.g. maximization of the area below the vacuum curve.

### LIST OF REFERENCES

100 breast pump assembly
110 breast pump
112 first button
114 second button
120 container assembly
122 breast shield
124 container
126 controller
130 program card
140 tube
150 interface
Cᵢ Cycle; i=1, 2, 3, 4
CTᵢ Cycle time or duration; i=1, 2, 3, 4
PDᵢ maximum vacuum strength; i=1, 2, 3, 4
VRP vacuum rise phase
VPP vacuum peak phase
VR₁P first vacuum release phase
VHP vacuum hold phase
VR₂P second vacuum release phase
HVP high pressure vacuum relaxation phase

What is claimed is:

1. A breast pump including a breast shield adapted to at least in part receive a breast of a lactating user, an aggregate for generating a cyclic suction profile according to vacuum curves for expressing milk from the breast, a milk container providing a reservoir for the expressed milk, a controller for controlling the aggregate
wherein the controller has a memory having stored a set of at least two vacuum curves, the vacuum curves being indicative of vacuum over time, each vacuum curve having a cycle phase and a relaxation phase, wherein vacuum curves with stronger maximum vacuum have shorter cycle phase duration.

2. The breast pump according to claim 1, wherein vacuum curves with stronger maximum vacuum have shorter vacuum curve duration.

3. The breast pump according to claim 1, wherein the controller orders the following sequence of phases
a vacuum rise phase (VRP) for decreasing the pressure from a maximum pressure to a minimum pressure being the maximum vacuum strength,
a vacuum peak phase (VPP) where the maximum vacuum strength is maintained
a first vacuum release phase (VR₁P),
a vacuum hold phase (VHP) holding a hold suction pressure for a vacuum hold time,
a second vacuum release phase (VR₂P) releasing the hold suction pressure to the maximum pressure and
a high pressure vacuum relaxation phase (HVP) defining the end of the cycle.

4. The breast pump according to claim 3, wherein at least 70% of any variation of the cycle phase duration is provided by the variation of the vacuum hold phase (VHP) and/or the vacuum peak phase (VPP) and/or the first vacuum release phase (VR1P).

5. The breast pump according to claim 3, wherein at least 70% of any variation of the vacuum curve duration is provided by the variation of the vacuum hold phase (VHP) and/or the vacuum peak phase (VPP) and/or the first vacuum release phase (VR1P) and/or the high pressure vacuum relaxation phase (HVP).

6. A set of vacuum curves for a breast pump, each vacuum curve being indicative of vacuum over time, each vacuum curve having a cycle phase and a relaxation phase, wherein vacuum curves with stronger maximum vacuum have shorter cycle phase length.

7. The set of vacuum curves according to claim 6, wherein vacuum curves with stronger maximum vacuum have shorter vacuum curve length.

8. The set of vacuum curves according to claim 6, wherein each vacuum curve has the following sequence of phases
a vacuum rise phase (VRP) for decreasing the pressure from a maximum pressure to a minimum pressure being the maximum vacuum strength,
a vacuum peak phase (VPP) where the maximum vacuum strength is maintained
a first vacuum release phase (VR1P),
a vacuum hold phase (VHP) holding a hold suction pressure for a vacuum hold time,
a second vacuum release phase (VR2P) releasing the hold suction pressure to the maximum pressure and
a high pressure vacuum relaxation phase (HVP) defining the end of the cycle.

9. The set of vacuum curves according to claim 8, wherein at least 70% of any variation of the cycle phase length is provided by the variation of the vacuum hold phase (VHP) and/or the vacuum peak phase (VPP) and/or the first vacuum release phase (VR1P).

10. The set of vacuum curves according to claim 8, wherein at least 70% of any variation of the vacuum curve length is provided by the variation of the vacuum hold phase (VHP) and/or the vacuum peak phase (VPP) and/or the first vacuum release phase (VR1P) and/or the high pressure vacuum relaxation phase (HVP).
